(19) United States
(12) Patent Application Publication
(10) Pub. No.: US 2023/0120525 A1
(43) Pub. Date: Apr. 20, 2023

(54) DETECTION DEVICE HOUSABLE OR INTEGRATED IN A FOOTWEAR

(71) Applicant: ESTEPS S.R.L.S., Rimini (IT)

(72) Inventor: Nidhal LOUHICHI, Rimini (IT)

(21) Appl. No.: 17/908,518

(22) PCT Filed: Mar. 5, 2021
(86) PCT No.: PCT/IB2021/051845

§ 371 Date: Aug. 31, 2022

(30) Foreign Application Priority Data
Mar. 6, 2020 (IT) 102020000004810

(51) Int. Cl. A61B 5/00; G01P 15/18; G01P 15/14; A61B 5/11

(52) U.S. Cl. CPC A61B 5/6807; G01P 15/18; G01P 15/14; A61B 5/1114; A61B 5/4561; A61B 5/7246; A61B 5/002; A61B 2562/0219; A61B 2562/04; A61B 2560/0214

(57) ABSTRACT

A detection device for detecting posture and movement of a user is provided. The detection device has a main body insertable or integrated in a footwear, a detection group embedded in the main body and having at least one detection member having at least one accelerometer element and at least one gyroscope element. The detection device has a power supply group embedded in the main body and having a generator organ of alternating current, and a generator organ of direct current connected to the generator organ of alternating current to receive and transform alternating current into direct current to supply electricity to the detection group.

The present invention relates to a detection device suitable for detecting the posture and movement of a user. In particular, according to the present invention, such a detection device is housable in a footwear. Or in an embodiment variant, again according to the present invention, said detection device is integrated in a footwear.

The object of the present invention is also a system for analyzing the posture and movement, which comprises at least one detection device, preferably two detection devices, and at least one external electronic device, such as a PC, a smartphone or a tablet.

In further detail of the present invention, the detection device is specifically applied in the field of medicine (for example within the realm of medical rehabilitation) and/or sports.

Solutions of detection devices and footwear comprising specific detection devices, the purpose of which is to detect a series of information associated with the posture and/or movement of a user, are known from the prior art.

Such known solutions however have a plurality of problems, which have affected the diffusion and use thereof.

Firstly, the known solutions are not able to constantly detect the desired information. Conventionally, the known products have a series of problems associated with the supply of electricity thereof, and therefore with the duration of the detections.

Secondly, the known solutions furthermore are particularly cumbersome, particularly with reference to the “electrical component” thereof. The greater the dimensions, the more the usability and aesthetics of the footwear for which they are provided, are affected.

It is therefore the object of the present invention to have a detection device which is housable or integrable in a footwear which meets the aforesaid needs of the field by providing an alternative to the known solutions, thus resolving the specific problems thereof.

In other words, the object of the present invention is to provide a detection device which ensures a constant and long-lasting detection of the posture and/or movement of a user so as to provide a complete picture, i.e. having an increased amount of information. In this way, the increased quantity of information collected may be taken advantage of by a treating physician or a trainer, but also simultaneously by the user him/herself according to needs.

According to the present invention, such an object is achieved by a detection device according to claim 1. Simultaneously, such an object is achieved by a system for analyzing posture and movement according to claim 10. Preferred embodiments of the invention are defined in the dependent claims.

The features and advantages of the above invention will become apparent from the following description, given by way of non-limiting example, according to the accompanying figures, in which:

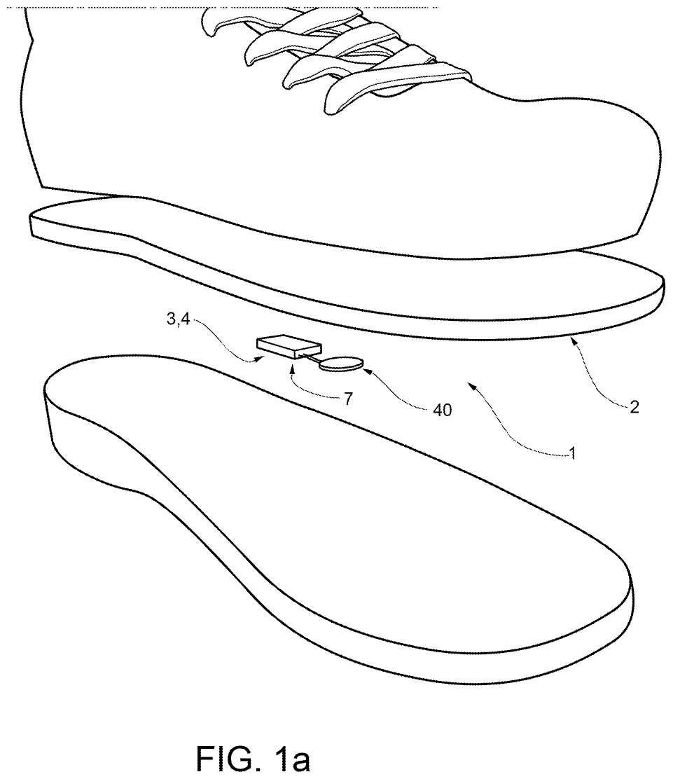

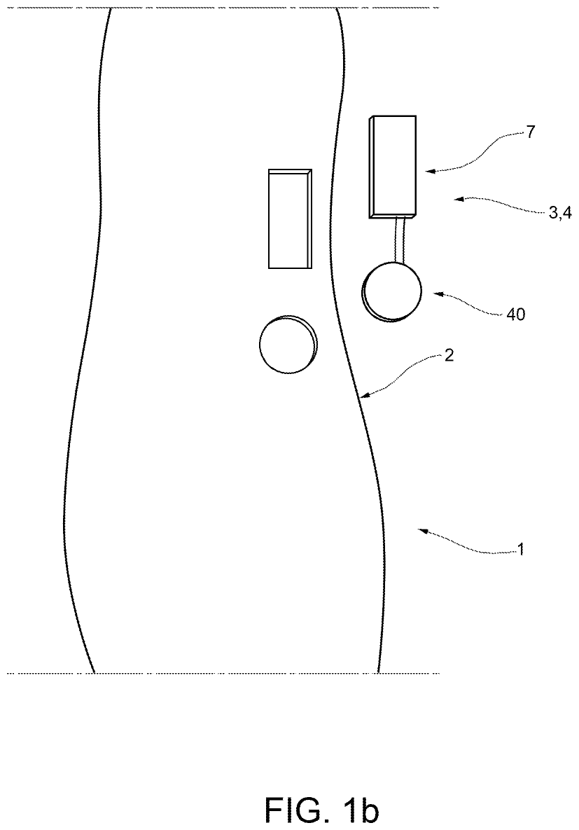

FIG. 1a shows a view with separate parts, of a footwear comprising a sole in which the detection device according to a preferred embodiment of the present invention is housed;

FIG. 1b shows a sectional view of the footwear in FIG. 1a;

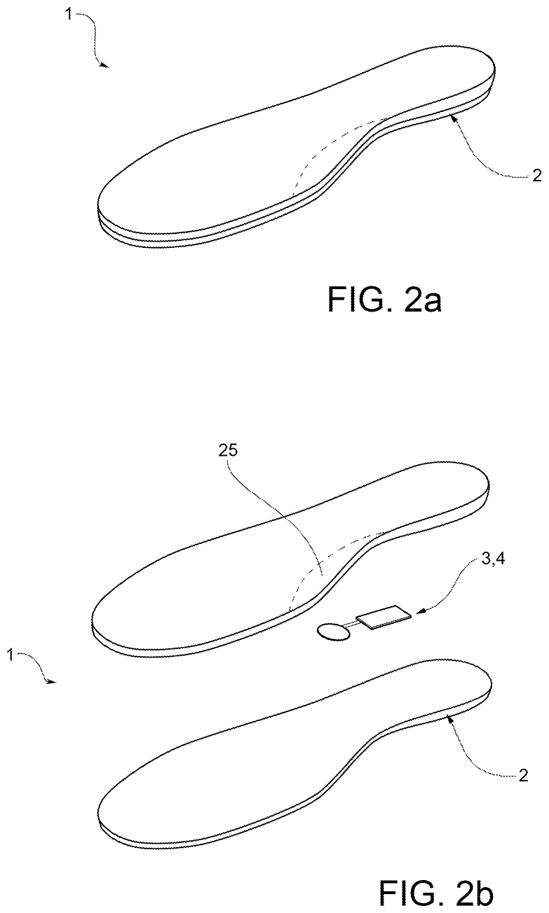

FIG. 2a shows a perspective view of the detection device in a preferred embodiment of the present invention, i.e. in the shape of an insole;

FIG. 2b is a perspective view, with separate parts, of the detection device in FIG. 2a;

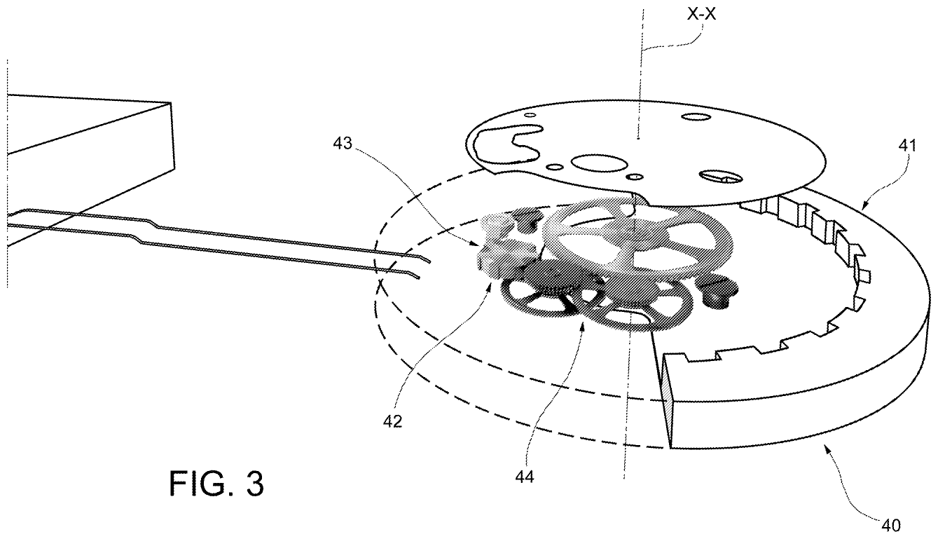

FIG. 3 is a perspective view, with separate parts, of some components comprised in the detection device according to the present invention;

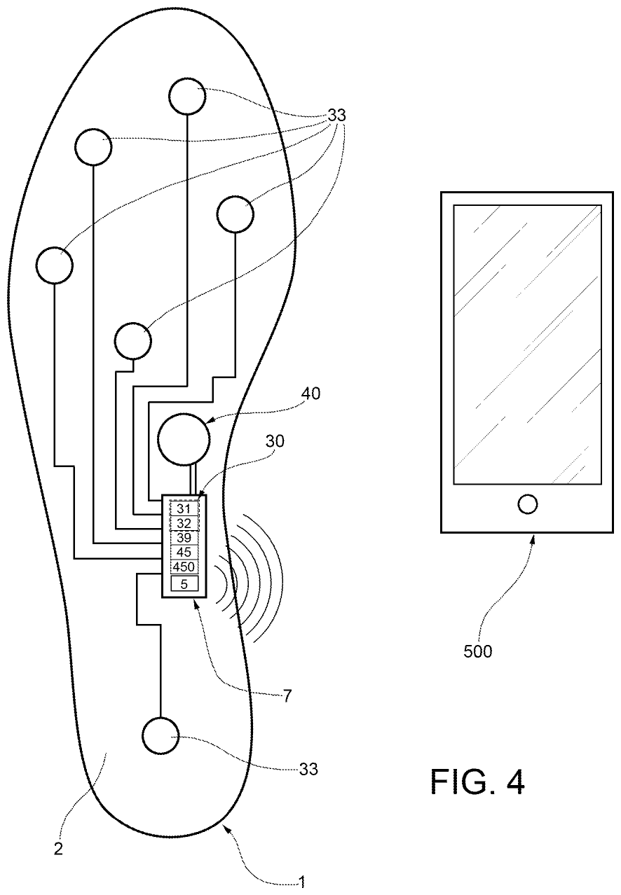

FIG. 4 shows a diagrammatical view of a system for analyzing the posture and movement of a user according to a preferred embodiment;

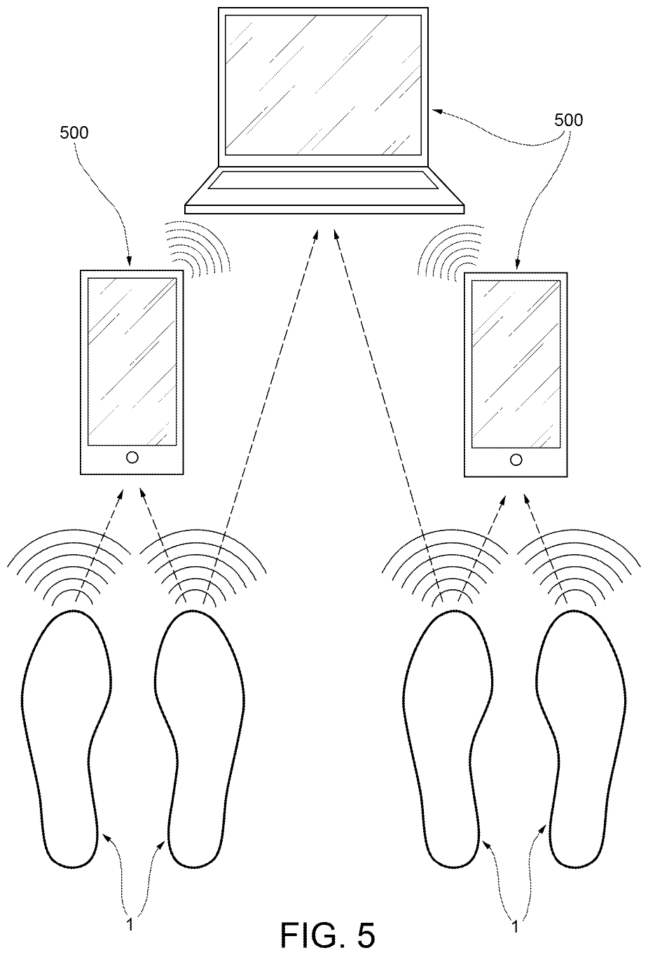

FIG. 5 shows a diagrammatical view of a system for analyzing the posture and movement of a user according to a further preferred embodiment.

According to the accompanying drawings, a detection device as a whole according to the present invention is indicated by reference numeral 1.

According to the present invention, the detection device 1 is suitable for being housed in a footwear.

Or, according to an embodiment variant of the present invention, the detection device 1 is integrated in a footwear.

It should be noted that the term footwear in the present invention is not intended as limited to any embodiment. Indeed, footwear means any type of garment suitable for protecting the foot of the user who is to perform a given activity.

Preferably, the footwear may be a shoe or a boot. According to the type of footwear, the detection device is suitable for detecting both the static and dynamic methods of use of the user.

In the present disclosure, detecting “posture” identifies static-type detections, while detecting “movement” identifies dynamic-type detections.

According to the present invention, the detection device 1 comprises a main body 2 which is insertable in a footwear.

According to a preferred embodiment, the main body 2 is a sole or insole which is insertable in the footwear.

According to the present invention, the detection device 1 comprises a main body 2 which is integrated in a footwear.

According to a preferred embodiment, the main body 2 is a sole or insole which is integrated in the footwear.

Preferably, with the exception of the features described below, the sole or insole does not have limiting features for the invention.

According to further embodiments, the main body 2 is integrated in other portions of the footwear, for example in the upper.

According to the present invention, the detection device 1 comprises a detection group 3 embedded in the main body 2.

Preferably, the specific object of the detection group 3 is to detect static and dynamic features of the user.

According to the present invention, the detection group comprises at least one detection member 30 comprising at least one accelerometer element 31 and at least one gyroscope element 32.

Preferably, the accelerometer element 31 is suitable for detecting certain dynamic features of motion.

Preferably, the accelerometer element 31 is of the type having three axes.

Preferably, the accelerometer element 31 is of the low-power type.

Preferably, the gyroscope element 32 is suitable for detecting both static features and dynamic features. In particular, the gyroscope element 32 is suitable for detecting the position, in particular a given angle of inclination, of the foot of the user. In particular, the gyroscope element 32 is suitable for detecting the direction of a movement of the foot of the user.

Preferably, the gyroscope element 32 is of the type having three axes.

Preferably, the gyroscope element 32 is of the low-power type.

According to the present invention, the accelerometer element 31 and the gyroscope element 32 are suitable for simultaneously constantly detecting, in such a way as to have the data of both detections for each moment in time.

According to a preferred embodiment, the detection member 30 further comprises a pressure measuring element 33, preferably a plurality of pressure measuring elements 33, which are suitable for detecting the presence of the user and/or the pressure the user puts on the detection device 1.

According to a preferred embodiment, each pressure measuring element 33 is of the type comprising resistive pressure sensors.

Preferably, each pressure measuring element 33 is thin, substantially extending planarly without thickness.

According to a preferred embodiment, the pressure measuring element 33 extends in such a way as to be positionable at all the homogeneous parts of the sole of the foot.

According to a preferred embodiment, the main body 2 (preferably in the sole or insole embodiment thereof) comprises pressure measuring elements 33 which are strategically positioned to have a sensor for each preferred area of the foot, in particular one in the heel, one in the outer plantar arch and four in the front area of the sole of the foot proximal to the toes.

According to a preferred embodiment, the accelerometer element 31, the gyroscope element 32 and the pressure measuring element 33 are suitable for simultaneously constantly detecting over time so as to have the data relating to each detection for each moment in time.

According to the present invention, the detection device 1 further comprises a power supply group 4 embedded in the main body 2.

The object of the power supply group 4 is to supply electricity to the detection group 3. Indeed, according to the present invention, the power supply group 4 is operatively connected to the detection group 3.

Preferably, the power supply group 4 comprises:

a generator organ of alternating current 40, comprising a rotary element 41 suitable for rotating freely when subjected to movement;

a generator organ of direct current 45, which is electrically connected to the generator organ of alternating current 40 that receives the alternating current and transforms it into direct current in order to supply electricity to the detection group 3.

According to the present invention, the power supply group 4 is as close as possible to the detection group 3.

Preferably, the generator organ of alternating current 40 is in a position which is proximal to the detection group 3.

According to a preferred embodiment, the generator organ of direct current 45 also comprises a battery element 450 suitable for storing the direct electrical current in order to pass it to the detection group 3.

Preferably, the battery element 450 is suitable for supplying electricity to the detection group 3 according to needs, regardless of the movement of the generator organ of alternating current 40.

According to a preferred embodiment, the detection group 3, the generator organ of direct current 45 are in a single box-like element 7 embedded in the main body 2.

According to a preferred embodiment, the generator organ of alternating current 40 is substantially disc-shaped, comprising a main axis X-X and an imaginary plane on which it extends substantially orthogonally to the main axis X-X.

Preferably, the generator organ of alternating current 40 has the substantial dimensions of a coin.

According to a preferred embodiment, the rotary element 41 has an eccentric shape with respect to said main axis X-X about which it rotates, in which said main axis X-X extends substantially vertically and/or substantially horizontally. Preferably, the positioning of the main axis X-X is relative to the ground surface on which the user moves. According to a preferred embodiment, said main axis X-X is inclined by a few degrees with respect to the vertical.

According to a preferred embodiment, the generator organ of alternating current 40 comprises a rotor 42 and a stator 43.

Preferably, rotor 42 is operatively connected to said rotary element 41.

According to a preferred embodiment, the generator organ of alternating current 40 further comprises transmission means 44 connecting the rotary element 41 with rotor 42.

According to a preferred embodiment, the detection device 1 further comprises a receiving/transmitting data group 5, which is operatively connectable to an external electronic device 500, such as a smartphone or a tablet.

Preferably, also the receiving/transmitting data group 5 is embedded in the main body 2.

Preferably, said operatively connected receiving/transmitting data group 5 is in turn supplied with electricity by the power supply group 4.

According to a preferred embodiment, the detection group 3 also comprises a storage unit 39 suitable for storing the data detected by the detection member 30.

According to a preferred embodiment, the detection group 3 and the power supply group 4, and preferably also the receiving/transmitting data group 5, are embedded in the main body 2 in a central/side position.

Preferably, the detection group 3 and the power supply group 4, and preferably also the receiving/transmitting data group 5, are positioned in an area which is substantially proximal to the plantar arch of the user of the sole or insole.

Indeed, the main body 2 in the shape of sole or insole preferably comprises a plantar arch reinforcement body 25, in which the detection group 3 and the power supply group 4 and the receiving/transmitting data group 5 are housed.

According to a preferred embodiment, said plantar arch reinforcement body 25 faces the inside of the foot of the user.

As mentioned, the object of the present invention is also a system for analyzing the posture and movement of a user, comprising at least one detection device 1, preferably two detection devices 1, and an external electronic device 500 that is operatively connectable to said detection device 1.

According to a preferred embodiment, the system for analyzing posture and movement comprises a pair of footwear, in which each footwear is inserted or integrated in a detection device 1.

Preferably, the external electronic device 500 is configured to receive data from each detection device 1 and is configured to correlate such information.

According to a preferred embodiment, the external electronic device 500 is a smartphone and/or a tablet and/or a workstation.

Preferably, the detection device 1 is suitable for transmitting information simultaneously to several external electronic devices 500.

According to a preferred embodiment, each external electronic device 500 is configured with specific programs (or apps) for collecting and managing the information detected by each detection device 1.

According to a preferred embodiment, the main body 2 in the form of insole comprises a preferably removable coating film suitable for protecting the insole and in certain cases the footwear itself, against the action of sweat, liquids and bacterial attacks.

Innovatively, the detection device according to the present invention achieves the preset object.

Advantageously, the detection device is suitable for constantly detecting data in such a way as to provide them to the user and/or a physician.

Advantageously, the supply of electricity is ensured by virtue of the self-powering of the detection device itself.

Advantageously, the battery serves to store a quantity of power such as to also ensure the supply of electricity to the detection device also in the absence of movements. Preferably, the detection device is suitable for sending the data collected also in the absence of movements, and therefore of self-powering.

Advantageously, the detection device is not very cumbersome and is versatile.

Advantageously, the detection device is housable or easily integrable in a footwear.

Advantageously, the detection device in the “insole” version is removable from a footwear to be insertable in another footwear.

Advantageously, the physician/trainer takes advantage of a constant flow of information from one or more users/patients, by means of the detection device.

Advantageously, the physician/trainer is free to do his/her work while operating remotely, without the need to be in the presence of the user.

Advantageously, the physician/trainer receives a very broad statistical sample of information.

Advantageously, the physician/trainer is able to guide the rehabilitation or physical training in light of any specific detections.

Advantageously, possible injuries to the lower limbs are easy to anticipate.

Advantageously, the user him/herself is able to monitor his/her daily routine and/or sports activities.

Advantageously, the status of the footwear is also monitorable, and for example the end of the life cycle thereof.

Advantageously, the status of the insole is also monitorable. Advantageously, the “electronic component” of the device is removable from the insole to be mountable in a new, specially-shaped insole.

Advantageously, the “mechanical component” of the detection device, i.e. the member that generates the alternating current, is housed in a clear housing which allows visually checking for possible malfunctions thereof.

It is apparent that those skilled in the art may make changes to the above-described invention in order to meet contingent and specific needs, all contained within the scope of protection defined by the following claims.

1-12. (canceled)

13. A detection device for detecting posture and movement of a user, comprising:
a main body, wherein the main body is an insole insertable into a footwear;
a detection group that is embedded in the main body and comprises at least one detection member comprising at least one accelerometer element and at least one gyroscope element; and
a power supply group that is embedded in the main body and operatively connected to the detection group to supply electricity to the detection group, wherein the power supply group comprises:
a generator organ of alternating current substantially disc-shaped, comprising a main axis (X-X), an imaginary plane on which the generator organ of alternating current extends orthogonally to the main axis (X-X), and a rotary element having an eccentric shape with respect to the main axis (X-X), suitable for rotating freely when subjected to movement, wherein the main axis (X-X) is inclined by a few degrees with respect to the vertical, wherein the generator organ of alternating current further comprises a rotor and a stator, said rotor being operatively connected to said rotary element; and
a generator organ of direct current, electrically connected to the generator organ of alternating current, that receives and transforms alternating current into direct current to supply electricity to the detection group.

14. The detection device of claim 13, wherein the generator organ of direct current comprises a battery element suitable for storing the direct current to pass the direct current to the detection group.

15. The detection device of claim 13, further comprising a group for receiving/transmitting data, which is embedded in the main body and operatively connectable to an external electronic device, wherein said group for receiving/transmitting data is supplied with electricity by the power supply group.

16. The detection device of claim 15, wherein said external electronic device is a smartphone or a tablet.

17. The detection device of claim 13, wherein the detection group and the power supply group are embedded in the main body in a central/lateral position so as to be aligned with a plantar arch of the user of the insole.

18. The detection device of claim 17, wherein the insole comprises a plantar arch reinforcement body, and wherein the detection group and the power supply group are housed in said plantar arch reinforcement body.

19. A system for analyzing posture and movement of a user, comprising at least one detection device according to claim 13, and an external electronic device operatively connectable to said at least one detection device.

20. The system of claim 19, comprising a pair of footwear, wherein the at least one detection device is inserted in each footwear of said pair of footwear.

21. The system of claim 20, wherein the external electronic device is configured to receive data from each detection device and correlate said data.
